(19) United States
(12) Patent Application Publication
(10) Pub. No.: US 2017/0281367 A1
(43) Pub. Date: Oct. 5, 2017

(54) 3D-PRINTED UNIBODY MESH STRUCTURES FOR BREAST PROSTHESIS AND METHODS OF MAKING SAME

(71) Applicants: 3DB RESOURCES LLC, Elma, NY (US); NTOPOLOGY INC., New York, NY (US)

(72) Inventors: Mary Jo Ketchum, Elma, NY (US); Bradley Rothenberg, New York, NY (US)

(21) Appl. No.: 15/501,711

(22) PCT Filed: Aug. 5, 2015
(86) PCT No.: PCT/US2015/043885

§ 371 Date: Feb. 3, 2017

Related U.S. Application Data
(60) Provisional application No. 62/033,584, filed on Aug. 5, 2014.

(51) Int. Cl. A61F 2/50; A61F 2/52

(52) U.S. Cl. CPC A61F 2/5046; A61F 2/52

(57) ABSTRACT

A breast prosthesis device and method for making same are disclosed. The prosthesis has an inner wall mesh having a first density. The inner wall mesh is configured to align with a chest wall of the user. The prosthesis has an outer wall mesh having a second density. The outer wall mesh configured to have an ideal shape for the user. The prosthesis may also have a band with a density greater or equal to the inner and outer wall meshes. The prosthesis has a central portion disposed in between the inner and outer wall meshes. The prosthesis can be generated using 3D scans of the user manipulated such that the resulting structure mimics human tissue.

### CROSS-REFERENCE TO RELATED APPLICATIONS

This application claims priority to U.S. Provisional Application No. 62/033,584, filed on Aug. 5, 2014, now pending, the disclosure of which is incorporated herein by reference.

### FIELD OF THE DISCLOSURE

The disclosure relates to three-dimensional (3D) printed unibody mesh structures for breast prostheses, in particular, to unique individualization of brassieres produced using additive manufacturing systems and techniques, which is also commonly referred to as 3D printing. The present disclosure accommodates for an anomalous breast shape of a user due to surgery or other natural shape differentiation.

### BACKGROUND OF THE DISCLOSURE

During surgery for breast cancers a certain amount of breast tissue may be removed from a user. Even a small amount of tissue removed creates a deficit that can't be overlooked, and causes anxiety. For example, it may become difficult to obtain satisfactory undergarments that will compensate for the deficit.

In addition, existing brassieres may create pressure from the brassiere structure, especially the chest/back band. This pressure is often a source of pain at the breast surgery site in many users, which may become very fatiguing and taxing as a day wears on. Straps and bands, while managing weight, volume and position, cause significant discomfort and often, pain, especially in post-surgical situations.

Traditional manufacturing techniques prefer uniformity in the manufactured goods being produced. Unfortunately, within a product design, specific portions of the design may require variability that would normally preclude use of single-piece manufacturing techniques. Accordingly, a product may be composed of several pieces or components joined together to accommodate these variations. For example, in clothing construction, two or more layers of fabric, plastic, leather, or other materials may be joined together along a seam, which stitches the different components together. Great care is taken during product design with respect to placement (e.g., inseam, center back seam, side seam, etc.) and type (e.g., plain, lapped, abutted, etc.) of the seams used to create a garment that fits properly. The result is a garment with several different component pieces joined together by several seams into a single article of clothing.

Previous additive manufacturing techniques, such as that described in U.S. Pat. App. Pub. No. 2014/0163445 use lattices to form a structure, however these lattices are unsuitable for breast prostheses and related garments. This technique creates stiffness in the lattice, for example, by increasing the number of layers or thickness of the lattice. In addition, the aforementioned lattice is a stiff structure—the lattice cannot move. As such, this design cannot account for the natural movement of the user, let alone provide dynamic support or a desired aesthetic appearance.

### BRIEF SUMMARY OF THE DISCLOSURE

One embodiment of the disclosure is a breast prosthesis device for a user. The breast prosthesis comprises an inner wall mesh having a first density. The inner wall mesh is configured to align with a chest wall of the user. The inner wall mesh may have fixed intersections.

The breast prosthesis device further comprises an outer wall mesh having a second density. The outer wall mesh is configured to have an ideal shape for the user. The outer wall mesh may have moveable intersections. For example, the outer wall mesh may have movable linkages to provide additional movement and flexibility.

The breast prosthesis device further comprises a band mesh having a density greater or equal to the first or second density of the inner wall mesh and outer wall mash, respectively.

The breast prosthesis device further comprises a central portion disposed in between the inner and outer wall meshes. The central portion may be filled with foam, batting, gel, or another suitable material. The central portion may be the same as the inner or outer wall meshes. The central portion may comprise several areas, each area having a density different than each other area.

One portion or all of the device may be formed from thermoplastic polyurethane and/or waterproof material. The device may further comprise a film applied to the inner wall mesh and/or outer wall mesh.

Another embodiment of the disclosure is a breast prosthesis device for a user comprising an inner wall mesh having a first density, the inner wall mesh having fixed intersections configured to align with a chest wall of the user; an outer wall mesh having a second density, the outer wall mesh having movable intersections configured to have an ideal shape for the user; a band mesh having a density greater or equal to the first or second density; and a central portion disposed in between the inner and outer wall meshes. The inner wall mesh is configured to align with the chest wall of the user based on two or more 3D scans of the user in one or more positions.

Another embodiment of the disclosure is a method for making a custom breast prosthesis for a user. The method comprises receiving, from a scanner, a first 3D scan of at least a portion of the user in a relaxed position. The method further comprises receiving, from the scanner, a second 3D scan of the portion of the user in a supported position. The supported, for example, may comprise the user's arms held straight up.

The method further comprises mapping, using a processor, one or more translation matrices corresponding to the first and second 3D scans.

The method further comprises analyzing, using the processor, the one or more matrices through warping and/or subdivision. Analyzing the one or more matrices may comprise warping the one or more matrices using a least-squares regression method. The method may further comprise analyzing the one or more matrices to identify regions of the user having scar tissue and adjusting the density of the structure based on the identified regions of the user.

The method further comprises generating a structure for the prosthesis based on the one or more analyzed matrices. The generated structure is flexible in three dimensions and/or porous. For example, the generated structure has moveable linkages to provide flexibility and/or the structure may comprise several areas, each area having a density different than each other area. The method further comprises generating a band in the structure, the band having a higher density than the structure. The band may be generated based on the shape of the user's upper torso, back, shoulder, or ribcage.

The method further comprises making, via additive manufacturing, a custom breast prosthesis for the user based on the generated structure and band. The prosthesis may be made using selective laser sintering. The method may further comprise applying a membrane to at least a portion of the prosthesis.

The method may further comprise receiving, from the scanner, a third 3D scan of an ideal portion of the user in a relaxed position; receiving, from the scanner, a fourth 3D scan of the ideal portion of the user in a supported position; mapping, using the processor, one or more translation matrices corresponding to the third and fourth 3D scans; and comparing, using the processor, the one or more translation matrices corresponding to the third and fourth 3D scans with the one or more translation matrices corresponding to the first and second 3D scans. The generated structure and generated band may be based on the compared translation matrices.

Embodiments of the present disclosure can supplement the breast tissue removed during a mastectomy with a prosthesis or garment that is comfortable, stable, secure and desirable in appearance. The appearance and dimensions of the prosthesis or garment are based on pre-and/or post-surgery scans as well as on individual preference, or in the absence of surgery, on contemporary scans.

The design of the prosthesis or garment, for example, may conform to the body's curvature, and can be flexible and still strong, without folding over, or requiring an underwire or other rigid material.

In one embodiment, straps and bands can be designed and produced to achieve support while distributing weight, avoiding painful pressure points, through the use of meshes and other geometries and materials that can be incorporated in 3D printing. In addition, a 3D printed prosthesis or garment as described herein can restore natural appearance and avoid an additional surgery for cosmetic reasons.

Another embodiment of the present disclosure is a breast prosthesis comprising an inner mesh portion having a first density, the inner mesh portion configured to align with a chest wall of the user; an outer mesh portion having a second density, the outer mesh portion configured to have an ideal shape for the user; and a band having a density greater or equal to the first or second density; wherein the inner mesh portion, outer mesh portion, and band are electronically designed based on two or more 3D scans of the user. In this way, the inner mesh portion and the outer mesh portion are connected and comprise the structure of the prosthesis.

### DESCRIPTION OF THE DRAWINGS

For a fuller understanding of the nature and objects of the disclosure, reference should be made to the following detailed description taken in conjunction with the accompanying drawings, in which:

FIG. 1 illustrates a perspective view of a 3D scan captured by a 3D sensor;

FIG. 2 illustrates a plan view of a 3D matrix around an anomalous breast shape;

FIG. 3 illustrates a plan view of a 3D subdivided matrix around an anomalous breast shape;

FIG. 4 illustrates an axonometric view of a 3D subdivided matrix around an anomalous breast shape;

FIG. 5 illustrates an elevation view of a 3D subdivided matrix around an anomalous breast shape;

FIG. 6 illustrates an axonometric view of a 3D subdivided matrix around an anomalous breast shape;

FIG. 7 illustrates an axonometric view of a 3D subdivided matrix around a breast cup;

FIG. 8 illustrates a side elevation view of a 3D subdivided matrix around a breast cup;

FIG. 9 illustrates a side elevation view of a 3D subdivided matrix around a breast cup;

FIG. 10 illustrates a perspective view of three different multidimensional fabrics;

FIG. 11 illustrates a perspective view of one multi-dimensional fabric;

FIG. 12 illustrates a perspective view of a multi-dimensional brassiere cup;

FIG. 13 illustrates a front elevation view of localized dimensional thickening of the multi-dimensional textile in areas of the cup that will touch scar tissue, and fill in what was removed during breast surgery, or what is lacking from a subjective point of view;

FIG. 14 illustrates a front perspective view looking at the inside of the brassiere showing the regions to be dimensionally thickened inside the cups;

FIG. 15 illustrates a side perspective view showing a bifurcating brassiere strap;

FIG. 16 illustrates a perspective view of the brassiere showing the multidimensional fabric around the cup and illustrates one embodiment of the present disclosure;

FIG. 17 illustrates a side elevation view of a matrix grid surrounding a normal breast shape;

FIG. 18 illustrates side elevation views of the matrix warping to adjust to other common breast shapes;

FIG. 19 illustrates a side elevation view of a matrix subdividing around regions of scar tissue in breasts after various surgery possibilities, including mastectomy and lumpectomy;

FIG. 20 illustrates the steps taken to generate the 3D custom fit brassiere;

FIG. 21 illustrates one type of infill for an embodiment of the present disclosure;

FIG. 22 illustrates another type of infill for an embodiment of the present disclosure;

FIG. 23 illustrates a first pass at calculating a mesh based on one or more 3D scans;

FIG. 24 illustrates a second pass at calculating a mesh based on one or more 3D scans;

FIG. 25 illustrates techniques for creating a central portion between an inner wall mesh and an outer wall mesh according to embodiments of the present disclosure;

FIG. 26 illustrates areas of varying density in a structure, where darker colors represent areas of lower density;

FIG. 27 illustrates a method of making a custom breast prosthesis according to one embodiment of the present disclosure; and

FIG. 28 illustrates a method of making a custom breast prosthesis according to another embodiment of the present disclosure.

### DETAILED DESCRIPTION OF THE DISCLOSURE

One embodiment of the present disclosure is a breast prosthesis device for a user. Although breast prostheses are described herein, the present disclosure may be used to generate other prostheses, such as prostheses that simulate other types of living tissue. The present disclosure may also be used to generate desired shapes or forms. At times, a user may require a re-balancing of a natural breast which has changed through aging to match the reconstructed breast which remains stable. In those cases, a user may desire and select a specific prosthesis/brassiere form.

Although the term prosthesis is used herein, some embodiments of the present disclosure may be used for aesthetic purposes, such as non-invasive tissue augmentation or other structural scenarios where a moveable, flexible, and life-like structure is desired. The device may be formed from thermoplastic polyurethane or another suitable material for additive manufacturing. The device may also be formed from biomaterials, such as polymer foams. Biomaterials may be well-suited for generating implantable prostheses capable of acting as a scaffold for adipogenesis.

The breast prosthesis device includes an inner wall mesh. The inner wall, for example, may refer to a wall of the device in contact with the human body, such as the chest. The term “inner” refers to the position of the device in relationship to the body. The term “inner may also refer to an inner circumferential portion of the device. The inner wall mesh may have a first density. The density may be determined through, for example, the thickness of the inner wall mesh, the structure of the inner wall, the material of the inner wall mesh, the density of the inner wall mesh, or a combination thereof. The inner wall mesh is configured to align with a chest wall of the user. For example, the inner wall mesh may be configured to substantially match the contour of the chest wall. However, the inner wall mesh may not be in direct contact with the chest wall. For example, a layer of fabric (such as the lining of a brassiere) may separate the inner wall mesh from the chest wall. In addition, the inner wall mesh may be configured to contact only certain portions of the chest wall, for example, portions of the chest wall that do not contain scar tissue or portions that can structurally support the device. In other embodiments, the inner wall mesh is configured to maximize contact with the chest wall while allowing the rest of the device to move with the body. The inner wall mesh may be configured to provide accommodation to sensitive regions of the chest/breast including scar tissue. The inner wall mesh may contain intersections in the mesh. These intersections may be fixed to provide additional rigidity and support.

The breast prosthesis also includes an outer wall mesh having a second density. The term “outer” refers to the position of the device in relationship to the body. The term “outer” may also refer to an outer circumferential portion of the device. The outer wall mesh may be in direct or indirect contact with the inner wall mesh. The second density may be the same as the first density. In most embodiments, the second density is less dense than the first density. The outer wall mesh may be configured to have an ideal shape for the user, for example, the idea breast shape, or a breast shape that closely resembles the other existing breast. The outer wall mesh may contain intersections in the mesh. These intersections may be moveable or slideable along the mesh in order to provide two or three dimensional movement of the outer wall mesh, and therefore adjoining portions of the device. For example, the outer mesh may contain moveable linkages to provide flexibility.

The breast prosthesis may also include a band. The band may be a mesh or the band may be a solid strand. The band can be formed from an elastic or non-elastic material. The band may be positioned in a similar location as an underwire on a traditional brassiere, or the band may be configured to provide support for the device in relation to the body. The band may have a density or stiffness greater or equal to the first or second density.

The breast prosthesis also includes a central portion disposed in between the inner and outer wall meshes. The central portion may also be a mesh and may be filled with foam, batting, gel, or a similar material. The central portion may comprise areas of varying density. The density may be determined by the type of mesh supplied in each area of the central portion by the thickness of the mesh, the structure of the central portion mesh, the material of the mesh, or a combination thereof The central portion may be more dense toward the inner wall mesh and less dense toward the outer wall mesh. The inner wall mesh, outer wall mesh, band, and central portion may be porous and/or permit airflow.

The breast prosthesis may also comprise a film applied to the inner wall mesh and/or outer wall mesh. The film may be a permeable membrane or a solid film. The film may have various thicknesses and textures. For example, a film applied to the inner wall mesh may be configured to detachably adhere to human skin. A film applied to the outer wall mesh maybe colored and shaped to appear like human breast tissue. Other anatomical features may be added to the film as desired. The film may be waterproof or resistant to puncture.

The inner wall mesh, outer wall mesh, band, and central portion may be design based on two or more 3D scan of the user.

Another embodiment of the present disclosure is a method for making custom breast prostheses for a user. One such embodiment is shown in FIG. 27. Although breast prostheses are described herein, the present disclosure may also be used to make prosthesis that simulate other types of tissue in a living being. Although the term prosthesis is used, some embodiments of the present disclosure may be used for aesthetic purposes, such as non-invasive tissue augmentation or other structural scenarios where a moveable, flexible, and life-like structure is desired.

The method 100 comprises receiving 101, from a scanner, a first 3D scan of at least a portion of the user in a relaxed position. The first 3D scan may be received and stored at a central repository, such as a server or database configured to store 3D scan data. The 3D scan may also be received 101 and stored on the 3D scanner or other suitable computing device. The 3D scan may comprise a point cloud, or other mathematical representation of a 3D space. The scan may include an image of a portion of the user, such as the chest wall. The term “relaxed” as used herein refers to the state where a user is standing up with the user's arms relaxed and to the user's side. Other positions may be used.

The method 100 further comprises receiving 103, from the scanner, a second 3D scan of the portion of the user in a supported position. The second 3D scan may be received 103 and stored at the same location as the first 3D scan. The term “supported” may refer to a state where the user is standing up with the user's arms extended upwards. Supported may also refer to a scan of the user wearing typical garments or brassieres that provide desired levels of support and shaping. Other positions and supports may be used.

The method 100 may further comprise mapping 105, using a processor (such as a server or computer executing specialized software), one or more translation matrices corresponding to the first and second 3D scans. For example, the translation matrices may include mathematical data capturing the correspondence between reference points identified in the first and second 3D scans. The membrane may be created from a coating material, such as a polymeric material. For example, silicone, polyurethane, polyepoxide, polyamides, or blends thereof.

The method 100 may further comprise analyzing 107, using the processor, the one or more matrices through warping and/or subdivision. For example, the data may be modified to manipulate or alter the matrices to simulate the scanned images in different positions and rotations. In one embodiment, warping involves using a least-squares regression method. The matrices may be subdivided into increasingly smaller shapes or grids in order to better approximate the desired shape, density, and movement of the device.

The method 100 may further comprise generating 111, using the processor, a structure for the prosthesis based on the one or more analyzed matrices and generating 115 a band in the structure. The structure may be a porous mesh. The structure may be generated in such a way that the structure is flexible in three dimensions. For example, this can be accomplished through non-fixed mesh intersections or movable linkages in the structure. The band may have a higher density or stiffness than the generated structure. The band may be generated 115 based on the shape of the user's upper torso, back, shoulder, or ribcage. The prosthesis may then may made 117 via additive manufacturing based on the generated 111 structure and band. A membrane may be applied 119 to the prosthesis.

In one embodiment, the method 100 may further comprise analyzing the one or more matrices to identify 109 regions of the user having scar tissue and adjusting 113 the density and/or movement of the structure based on the identified regions of the user.

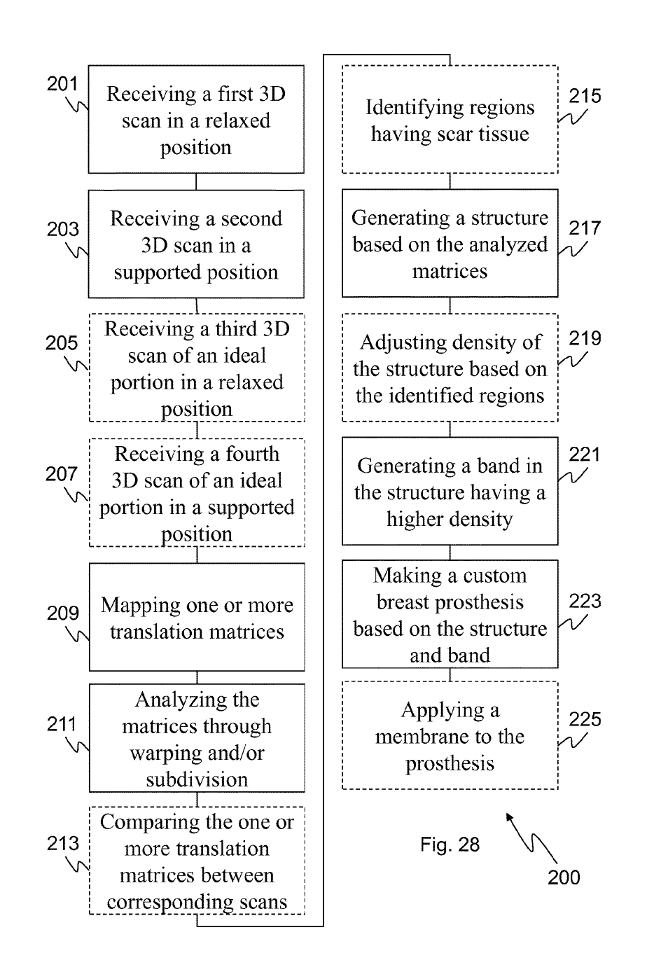

In another embodiment, as shown in FIG. 28, the method 200 may further comprise receiving 205, from the scanner, a third 3D scan of an ideal portion of the user in a relaxed position and receiving 207, from the scanner, a fourth 3D scan of the ideal portion of the user in a supported position. The third and fourth 3D scan may capture an ideal portion of a body, for example, the unaffected breast. In other words, the ideal portion of the body may be the desired final shape of the prosthesis. The processor may be used to map 209 one or more translation matrices corresponding to the third and fourth 3D scans. The processor compares 213 the one or more translation matrices corresponding to the third and fourth 3D scans with the one or more translation matrices corresponding to the first and second 3D scans. As such, the generated structure and band may be based on the compared translation matrices in order to create a prosthesis that matches an unaffected breast.

In accordance with various embodiments of the disclosure, unique unibody individualization of brassieres produced using 3D printing systems and methods are described that overcome the herein aforementioned disadvantages of the heretofore-known methods and systems of this general type and that provide for anomalous breast shapes due to surgery, as well as naturally occurring differentiation. These entirely and exclusively unique brassieres are designed and produced for the anomalous breast shape(s) of single individuals. Moreover, the disclosure, by virtue of being produced through 3D printing, will yield a brassiere that is lighter, more comfortable, and uniquely more effective in compensating for absent breast tissue. The present disclosure improves upon and surpasses the heretofore-known methods and systems, such as silicon prostheses and/or custom-molded cups, as a result of 3D scanning and printing systems. More specifically, the described embodiments provide conformity to a user's unique body dimensions including, but not limited to chest circumference, skeletal structure, posture, existing or pre-existing breast volume and dimensions, et. al.

The process of manufacturing each uniquely designed and produced 3D brassiere may include taking at least one 3D scan, mapping of the scans using 3D matrices, analysis of the 3D matrices through warping and subdivision, generating cups using multi-dimensional printable fabrics, generating a band that is custom fit to the shape and posture of the subject, and printing the multi-dimensional fabric using Selective Laser Sintering (SLS) or other suitable additive manufacturing process. Warping and subdivision help to adjust the prosthesis for fit and to highlight potential regions with scar tissue, particularly if the user previously underwent surgery or the breast includes areas to be normalized. The generated prosthesis may be dimensionally thickened or thinned in areas to be normalized or in surgically sensitive locations to improve airflow and feel by controlling fabric contact points in the sensitive skin area. By projecting curves in the shape of the straps onto the 3D model generated from the 3D scan, the band being generated may also be custom fit to the shape and posture of the upper torso, back, shoulder, and/or ribcage. The results of this process are two-fold: first, a unique unibody individualization brassiere can be printed as a full brassiere, or alternatively, printed in multiple pieces, i.e., a unique harness and a unique cup, that are fit completely and exclusively for a single user.

One feature that distinguishes this brassiere from all others is that unlike prior brassieres, the unique unibody individualization 3D brassiere is entirely unique to the user, overcoming the disadvantage of prior remedies for anomalous breast shapes, which consist of utilization of standardized brassieres, including the harness or structure, sometimes altered, with cups that are either standard sizes or customized.

Additive manufacturing (i.e., 3D-printing) allows traditionally separate portions of a product to be made without seams or welds. While additive manufacturing techniques can eliminate some of the seams between similar components in a product, some required variability cannot be eliminated by existing techniques that assume fabric uniformity. Moreover, when producing a product using an additive manufacturing process, the product design itself is often changed by the very materials used to manufacture the design. Thus, traditionally, component materials are selected by the manufacturer afterwards to match a desired design. Alternatively, if use of a particular material is desired, the design must incorporate that material from the beginning of the design process. Accordingly, any existing seamless product produced using currently available additive manufacturing techniques is limited to a single material selected for exhibiting properties consistent with the target design.

Another example of additive manufacturing is stereolithography technique, which relies on a bottom-up, layer-by-layer approach. This process usually involves a platform (substrate) that is lowered into a photo-monomer (photopolymer) bath in discrete steps. At each step, a laser is scanned over the area of the photo-monomer that is to be cured (polymerized) for that particular layer. Once the layer is cured, the platform is lowered a specific amount (determined by the processing parameters and desired feature/surface resolution) and the process is repeated until the full 3D structure is created.

Other 3D printing methods include Fused Deposition Modeling (FDM), Inkjet Deposition (U), Layered Object Manufacturing (LOM), Inkjet Binding (IB), Laser Powder Forming (LPF), Solid Ground Curing (SGC), Selective Laser Sintering (SLS), and Electron Beam Melting (EBM).

Embodiments of the present invention may use dynamic cellular microstructure designs. These designs customize production in an additive manufacturing construction. For example, using this technique, a seamless mesh structure may be electronically generated from an input shape (i.e., a chest wall of a user or another body part), based on available scans and/or surface designs. The mesh structure may be supplemented with curvature data derived from the input shape. This technique allows for the design and redesign of a base shape and/or group of base shapes within the seamless mesh. This enables customization in localized areas of the seamless mesh. For example, the design of a base shape within the seamless mesh of a breast prosthesis may allow for areas of varying density and movement. The seamless mesh may also be retopologized according to localized feature attractor points. For example, localized feature attractor points may include landmarks on the chest wall or other landmarks on the body, whether anatomical or electronically designated. Base shape redesign may include cellular replication, subdivision, growth, and/or modification to adjust variable material properties. Modification changes relative opacity, stretch, drape, compressive strength, plasticity, yield strength, resilience, and Poisson's ratio specific to geometry of a base shape. Each base shape can also exhibit modifiable isotropic or anisotropic properties.

In some embodiments, a method of manufacturing a seamless mesh may include obtaining at least one 3D scan of a 3D surface and/or a surface design to be at least partially covered by the seamless mesh, demarcating a portion of the obtained 3D scan and/or surface design as an input shape for the seamless mesh, identifying at least one base shape for use in creating the seamless mesh on the input shape, replicating the at least one base shape to cover the input shape with replicated base shapes that form the seamless mesh, and/or modifying the at least one base shape in localized areas of the seamless mesh based on relative proximity curvature of the input shape. In some embodiments, the modifying the base shape may include changing at least one of opacity, thickness, stretch, drape, and size of the base shape. In one embodiment, the base shape represents a combination of material (textile) and rules (template). For example, one embodiment of the present invention may include a seamless mesh for a custom brassiere having support straps around the shoulder or rib cage.

Modifications to the base shape can produce variable material properties. For example, parts of the base shape can thicken or thin, new connections can be added or removed within the base shape, and the method a base shape connects to its neighbor can change from interlocking to interconnecting. These changes can also alter a material's opacity, stretch, drape, as well as the final materials yield strength, Poisson's ratio, and/or compressive strength. The chosen base shape can also have isotropic or anisotropic properties, where isotropic properties of a base shape are the same in all orientations and anisotropic properties exhibit different properties depending on the orientation of the base shape.

In some embodiments, the base shape is a space filling polyhedral. For example, depending on the desired application, a base shape may be 2D and/or 3D (polygon/polyhedron). Thus, the mesh structure may be a framework which contains a regular, repeating pattern, wherein the pattern can be defined by a certain unit cell. A unit cell is the simplest repeat unit of the pattern. Thus, the mesh structure may be defined by a plurality of unit cells. The unit cell shape may depend on the required stiffness and can for example be triclinic, monoclinic, orthorhombic, tetragonal, rhombohedral, hexagonal or cubic.

As used herein, the terms “3D scan” and “3D surface” may or may not be used interchangeably depending on context and typically refer to a method to capture a three dimensional representation of an object. More specifically, the term “3D scan”, without additional context, refers to a method to capture three dimensional points in the real world through a device, such as a camera/scanner that can understand height, width and depth of an object being scanned and may also identify other parameters of the scanned object including color. A reconstruction of the space that is scanned is possible by generating a mesh from these points. Comparatively, the term “3D surface” may refer to a two-dimensional topological manifold in 3D space. Each point on the surface can be represented by a two-dimensional coordinate. Surfaces can be open, and have a boundary (ex. a plane), or closed (ex. a sphere). The term “input shape” refers to a 3D shape with any topology that is input either from an existing 3D model or is created from 3D scan data. Similarly, the term “base shape” refers to the combinational pair of a template cell and a textile cell, where the template cell has rules for growth and the textile cell gets updated parametrically based on the paired template cell.

The terms “matrix”, “shell”, and “mesh” may or may not be used interchangeably depending on context and typically refer to either a surface or structure readily recognized as a Cartesian way of representing object geometry using vertices, edges, and faces. A vertex has a specific Cartesian coordinate in relative space, edges connect any two vertices, and a face represents a closed set of edges. Usually each face consists of triangles or quadrilaterals, but any number of sides greater than three is possible. These terms may also refer to an openwork fabric, structure; a net, or network where individual cords, threads, or wires surrounding the spaces cover an input shape. The terms “remote” and “local” generally are not interchangeable and specifically reference to two distinct devices, but may not necessarily describe relative proximity depending on context. For example, items may be stored on a local client datastore and a remote server datastore, but the local datastore may actually be farther away if the local client datastore is actually maintained in cloud storage associated with the client.

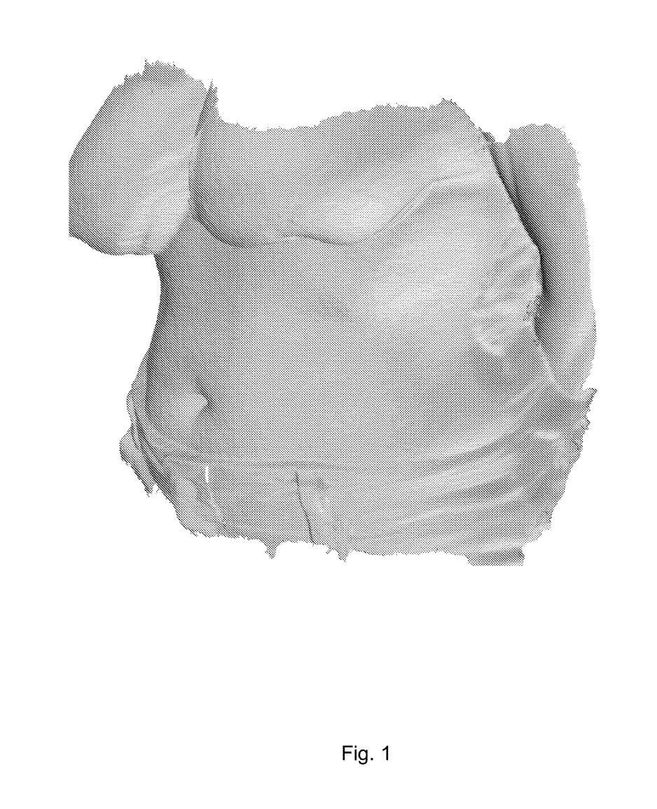

Referring to FIG. 1, a perspective view of a 3D scan captured by a 3D sensor. In this embodiment, a 3D scan is taken from a mobile device, utilizing a 3D depth sensor, of the breasts in the relaxed position. A 3D scan may also be taken with the user holding her arms straight up, resulting in the breasts being akin to the desired supported position. Other scans in contemporary supported positions are also possible. A 3D mesh is generated digitally from the point-cloud data resulting from the at least one 3D scan. This captures the shape of both breasts, relaxed and supported including the unique dimensions of each user, including but not limited to chest circumference, skeletal structure, posture, existing or pre-existing breast volume and dimensions. This data will inform the creation of the 3D Brassiere Shape, leading to a 3D brassiere that is completely unique to the user in size and fit.

The 3D scan should also be taken before surgery when possible, to accurately represent the breast's shape, to optimize brassiere fit post-surgery.

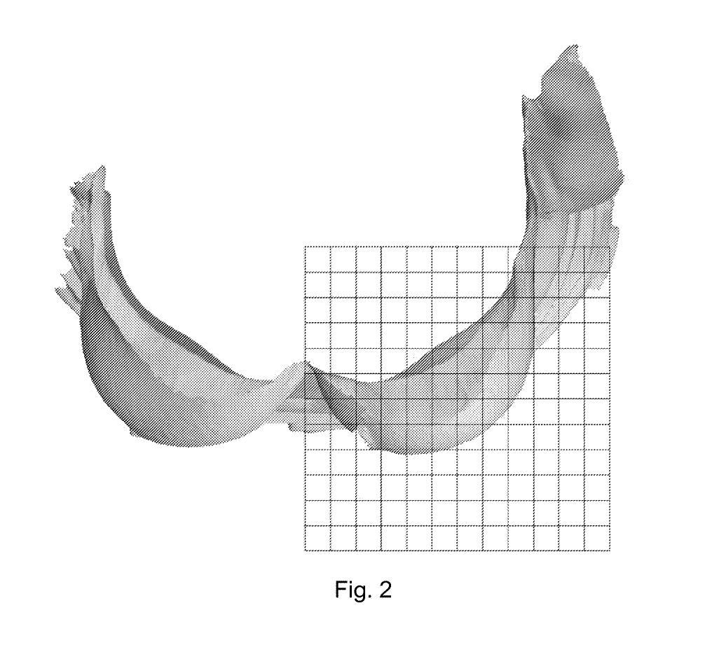

Referring to FIG. 2, a plan view of a 3D matrix around an anomalous breast shape. A 3D Matrix, or 3D grid, is applied in this embodiment. The 3D grid is used to locate the scar site where tissue was surgically removed from the breast. Scars are areas of fibrous tissue that replace normal skin after injury. Specifically, scar tissue is formed following an injury by connective tissue (non-elastic collagen fibers) that replaces normal soft functional tissue.

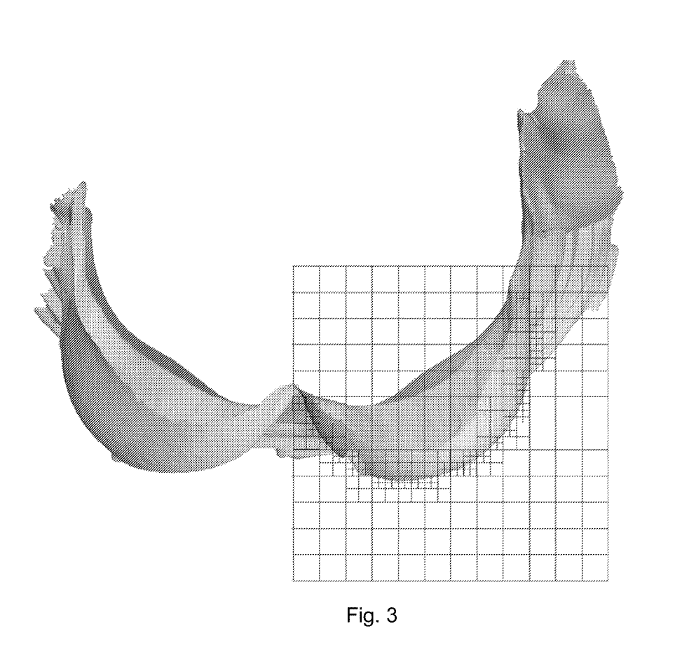

Referring to FIG. 3, a plan view of a 3D subdivided matrix around an anomalous breast shape. The 3D matrix, or 3D grid is subdivided, to increase the resolution around the areas with scar tissue. As such the matrix will be capable of filling in, external to the surgical site, the space resulting from removal of breast tissue, and compensating for the anomalous breast shape.

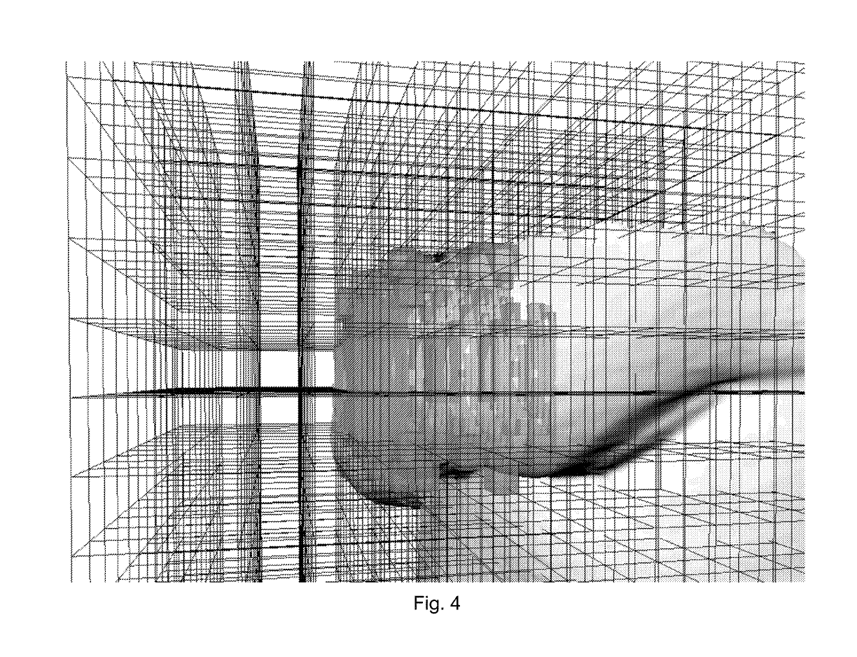

Referring to FIG. 4, an axonometric view of a 3D subdivided matrix around an anomalous breast shape. This Embodiment shows a 3D view of the subdivided 3D matrix, surrounding the breast with scar tissue. This 3D matrix is used for the basis of transformation to locate the scar tissue areas in the supported breast position that is optimal for the 3D Brassiere.

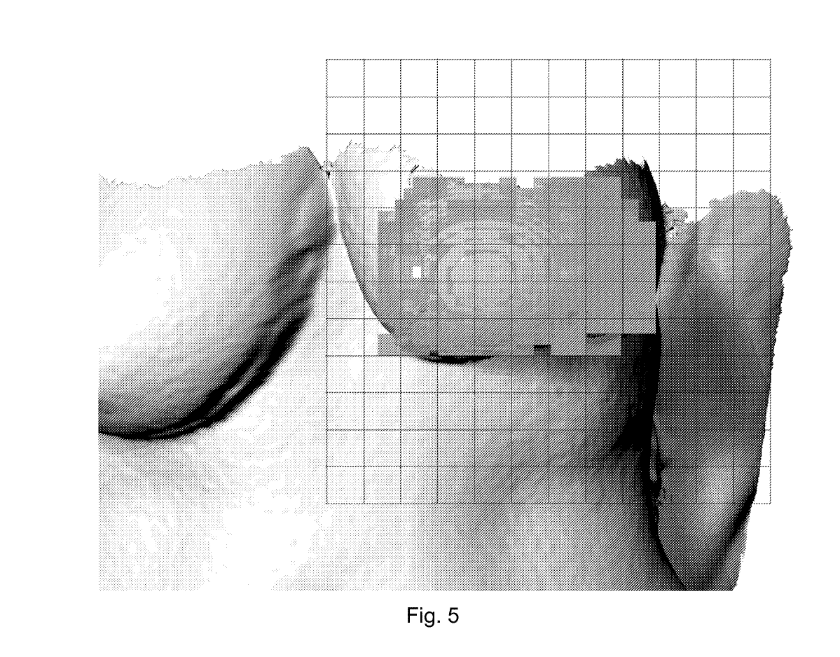

Referring to FIG. 5, an elevation view of a 3D subdivided matrix around an anomalous breast shape. A front elevation view of the 3D matrix surrounding the breast that underwent surgery, in this embodiment shown represented digitally as a 3D mesh.

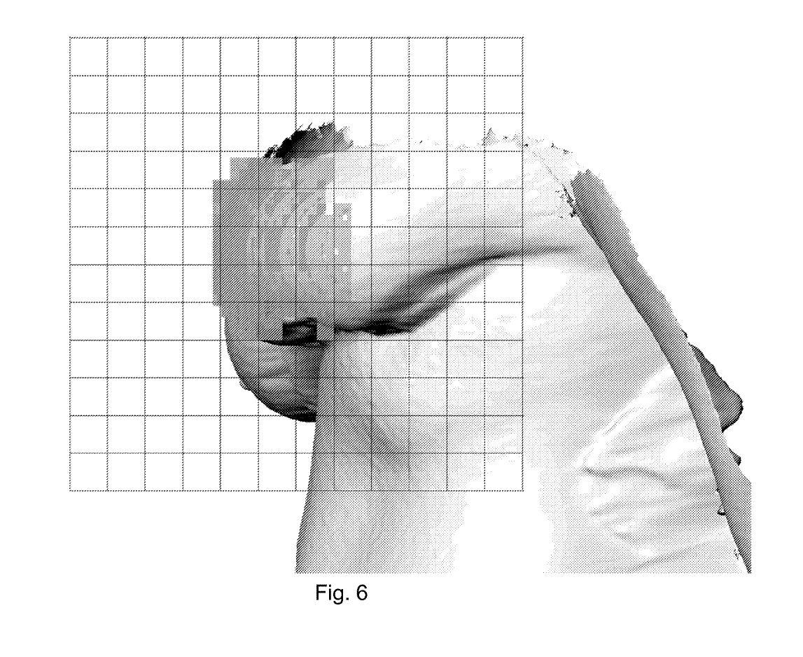

Referring to FIG. 6, an axonometric view of a 3D subdivided matrix around an anomalous breast shape. A side elevation view of the 3D matrix surrounding the breast that underwent surgery, in this embodiment shown represented digitally as a 3D mesh.

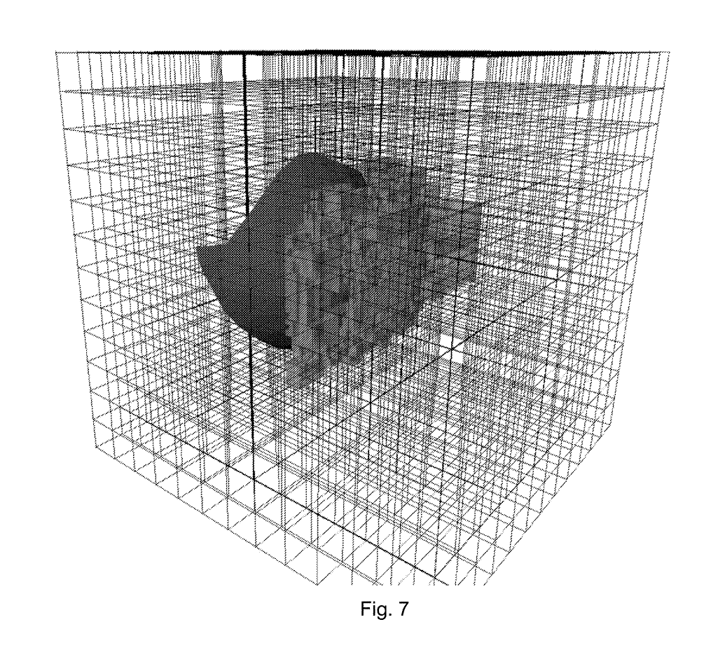

Referring to FIG. 7, an axonometric view of a 3D subdivided matrix around a breast cup. The 3D matrix, with the subdivided grid cells representing the scar tissue areas, and shape of the brassiere transforms the shape of the 3D Brassiere cup. Material is added or removed from the brassiere cup shape to fit the unique shape of the breast that could have undergone surgery for breast cancer.

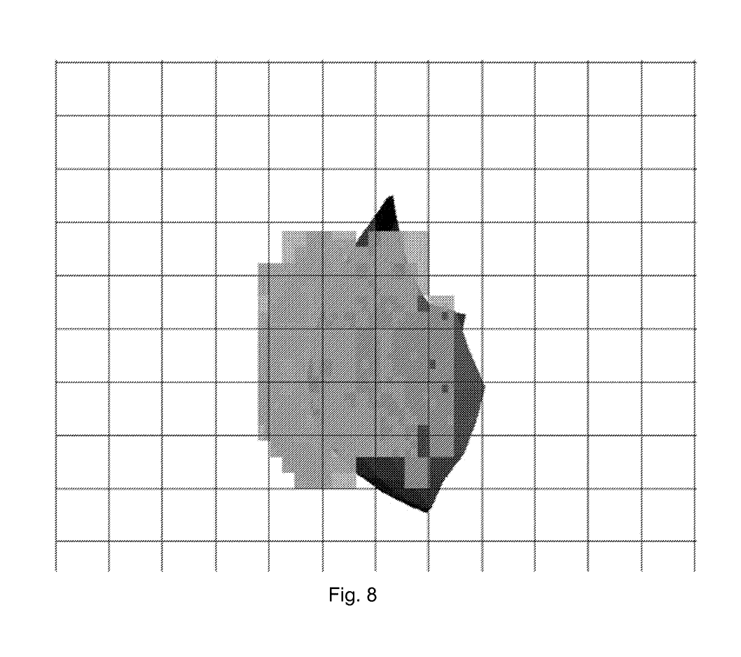

Referring to FIG. 8, a side elevation view of a 3D subdivided matrix around a breast cup. A side elevation view of the subdivided 3D matrix applied to the brassiere cup, representing the shape of the breast in this embodiment.

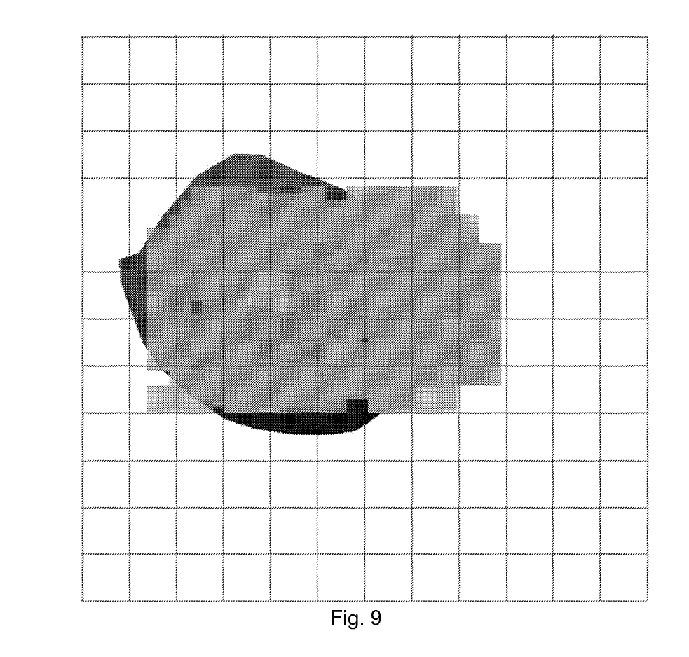

Referring to FIG. 9, a side elevation view of a 3D subdivided matrix around a breast cup is shown in accordance with at least one embodiment. A front elevation view of the subdivided 3D matrix applied to the brassiere cup, representing the shape of the breast in this embodiment.

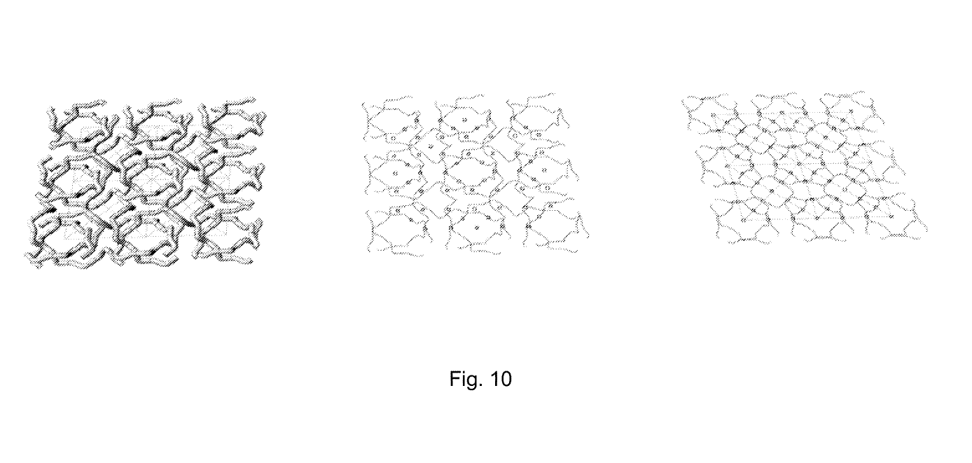

Referring to FIG. 10, a perspective view of different multidimensional fabrics is shown in accordance with at least one embodiment. A 3D textile is generated, creating a material that has varying flexibility, the first fabric shown can flex in the 3 directions, x and y, horizontally, and z, vertically. The second fabric shown can flex in 2 directions, X and Y horizontally. The third fabric shown can flex in one direction, X horizontally. These textiles are used in all parts of the Brassiere, changing the amount of stretch in the different parts of the brassiere, cup, shoulder straps, and back straps. Pressure of the brassiere structure, especially the chest/back band, is often a source of pain at the breast surgery site in many users, that becomes very fatiguing and taxing as a day wears on. Additionally, the brassiere straps' weight bearing role is concentrated on a narrow portion of the shoulders. The characteristics of the 3D textile herein described, will allow for a more balanced distribution of the breasts' weight and mass, thereby alleviating the pressure points, both natural, and in many cases, surgically induced.

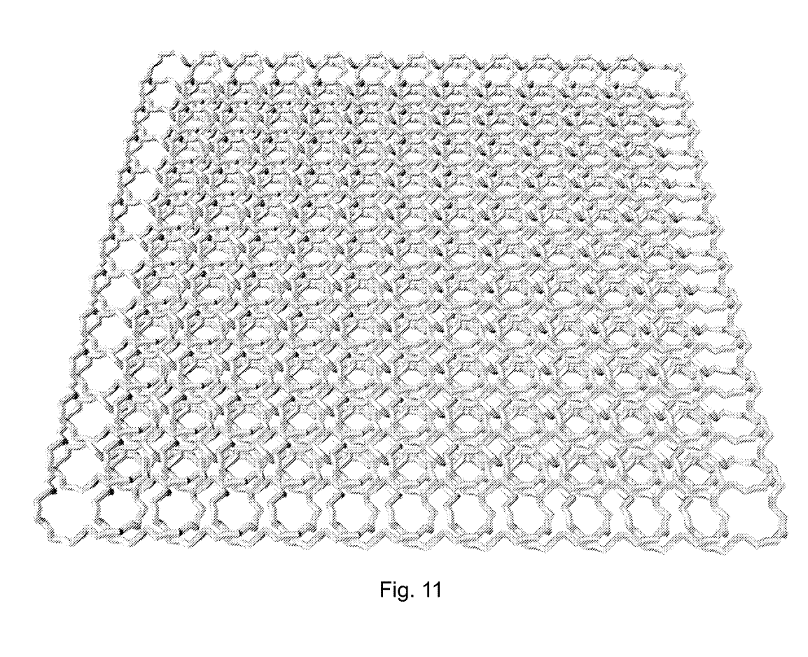

Referring to FIG. 11, a perspective view of one multi-dimensional fabric is shown in accordance with one embodiment. This embodiment shows the 3D textile in a flat and even layout. This textile is optimized for the process of 3D printing, and could not be made through any other textile methods. 3D printing, specifically SLS or other suitable additive manufacturing process, is a production method for the complex geometry of the interlocking units that vary in size and dimension throughout the brassiere. The textile will provide stable and secure support, while being wearable for a normal time period of the day and be of such quality and durability as to withstand frequent launderings.

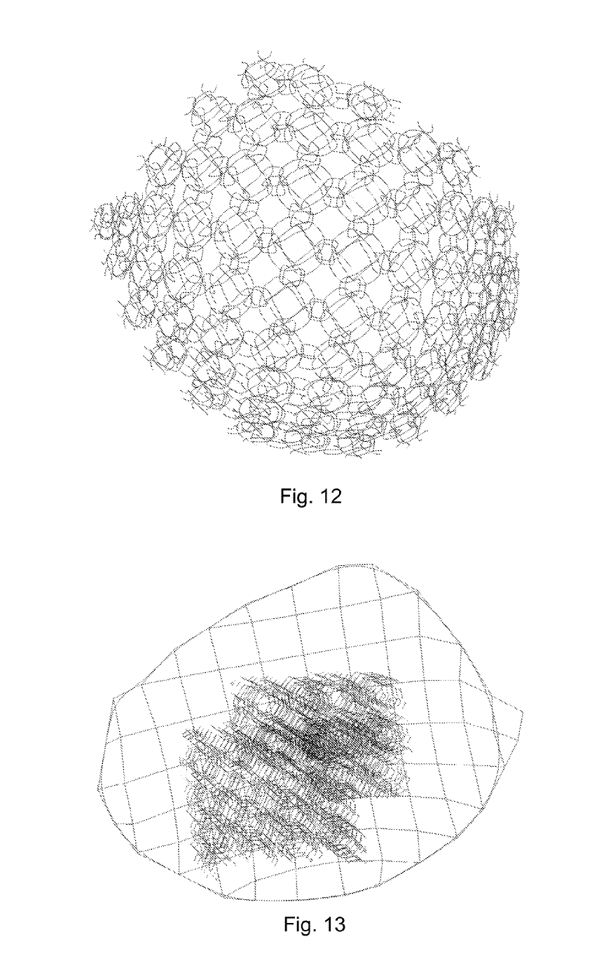

Referring to FIG. 12, a perspective view of a multi-dimensional brassiere cup is shown in accordance with at least one embodiment. In this embodiment, the 3D textile is applied to the cup shape, determined from the subdivided 3D matrix.

Referring to FIG. 13, a front elevation view of localized dimensional thickening of the multi-dimensional textile is shown in areas of the cup that will touch scar tissue, and fill in what was removed during breast surgery in accordance with at least one embodiment. In this embodiment, the properties of the 3D textile allow a 3D thickening, creating a mesh that fills in to the shape of the breast. This interlocking mesh is soft to touch the delicate scar tissue in the areas of the breast that underwent surgery. The mesh also allows for sweat and air to move through the open cells, creating a breathable material surrounding the breasts.

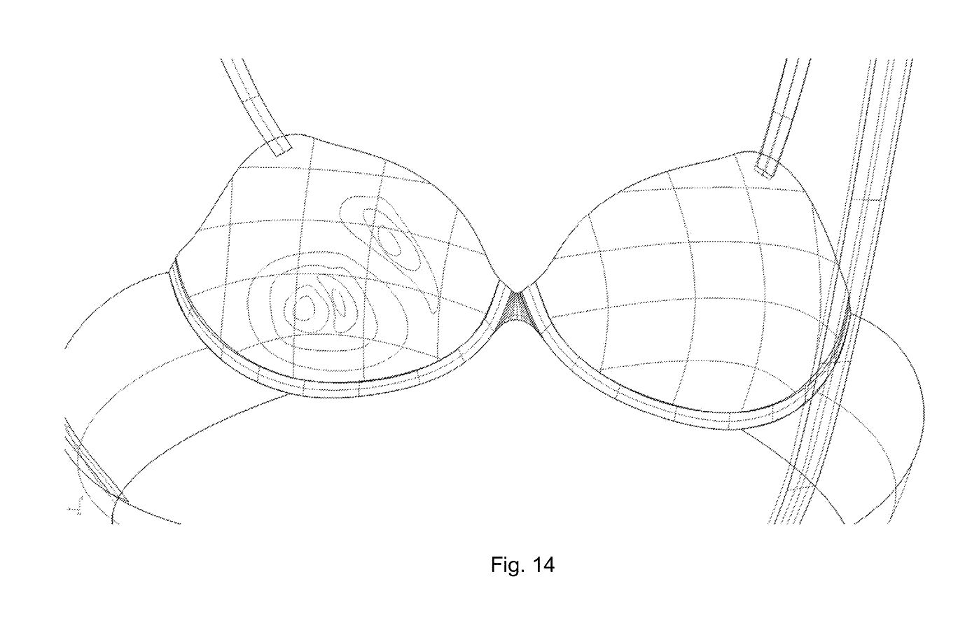

Referring to FIG. 14, a front perspective view looking at the inside of the brassiere showing the regions to be dimensionally thickened inside the cups is shown in accordance with at least one embodiment. An elevation view showing the areas inside of the brassiere that are thickened to fill in the parts of the breast removed during surgery. The thickening is custom per brassiere and per surgery. Design of the 3D printed brassiere is planned so that the support structure's material will conform to the body's curvature, be flexible and still strong, without folding over, or requiring an underwire or other rigid material.

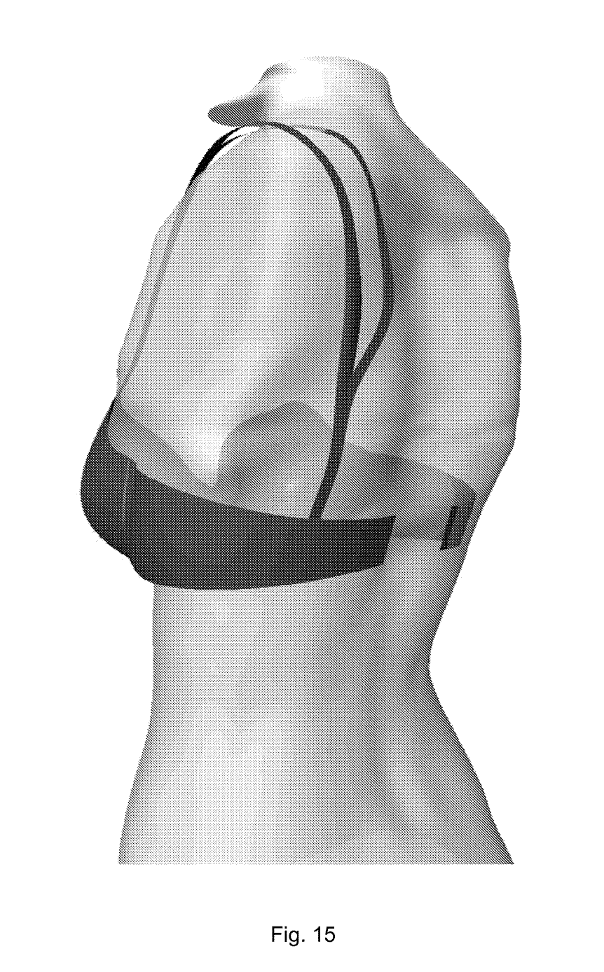

Referring to FIG. 15, a side perspective view of a bifurcating brassiere strap is shown in accordance with at least one embodiment. In this embodiment, the brassiere straps bifurcate and split around the shoulder blade, creating more area to support the breasts. The 3D printed textile allows for varying stretch throughout the straps, and allow for the weave of the textile to define the shape of the brassiere. Design of the 3D printed brassiere is planned so that the support structure's material will conform to the body's curvature, be flexible and still strong, without folding over, or requiring an underwire or other rigid material. A custom band is fit to the shape and posture of the upper torso, back, shoulder, and/or ribcage by projecting curves onto the 3D model generated from the 3D scan.

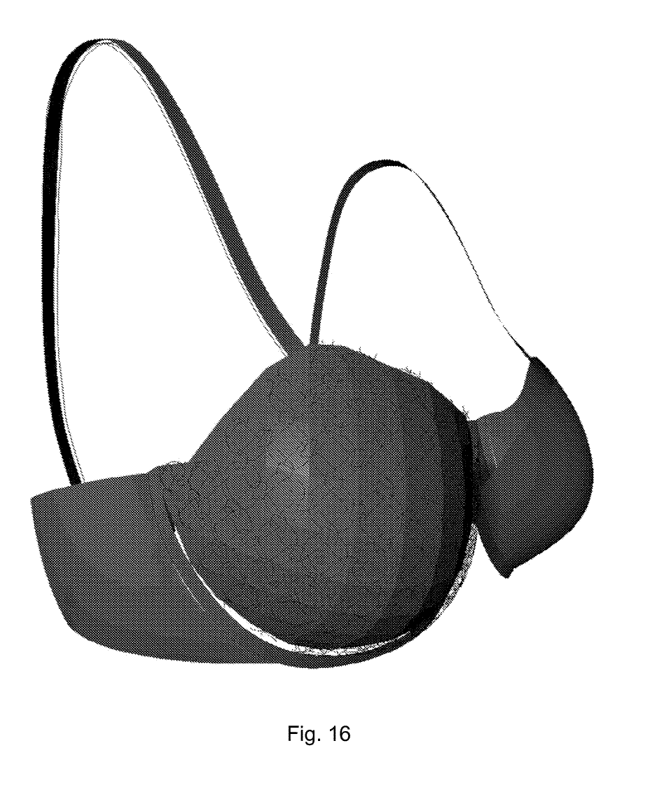

Referring to FIG. 16, a perspective view is shown of the brassiere showing the multi-dimensional fabric around the cup and illustrates the unique unibody individualization of 3D printed brassiere in accordance with at least one embodiment. An embodiment of the brassiere with the 3D textile filling in the shape of the cup to fit specifically to the breast. The value to users of 3D printed brassieres that will give the user the appearance the user previously enjoyed of the user's own natural body, without the need for breast reconstruction cannot be overstated.

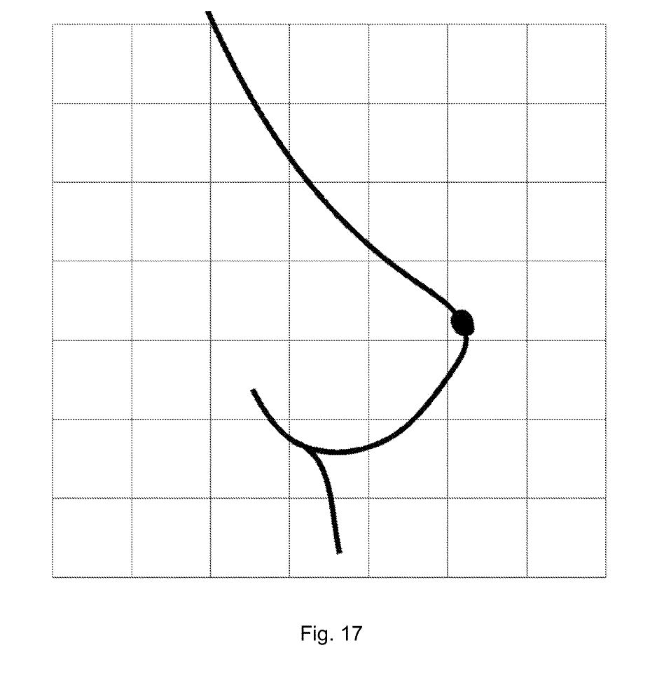

Referring to FIG. 17, a side elevation view of a matrix grid surrounding a normal breast shape is shown in accordance with at least one embodiment. This embodiment shows a matrix grid surrounding an idealized, supported breast shape. This grid forms the basis of transformation from the scan data, accurately showing regions that underwent surgery, or missing regions on which the multi-dimensional fabric can fill in.

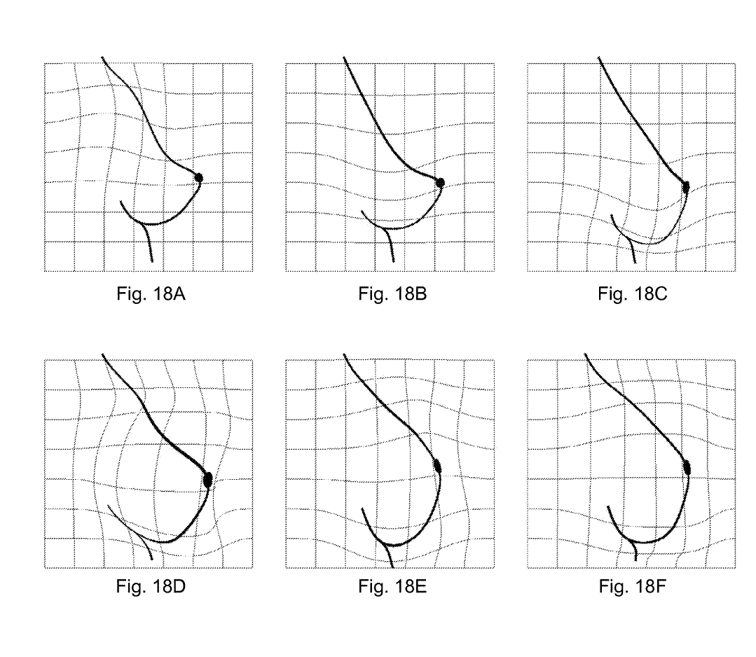

Referring to FIG. 18, side elevation views of the matrix are shown warping to adjust to other common breast shapes in accordance with at least one embodiment. In this embodiment, the matrix grid is warped to show the transformation from various sagging breast shapes. This warped grid transforms to the idealized supported position in order to accurately locate the parts of the cup that need to be filled in to touch the regions of the breast with scar tissue.

Referring to FIG. 19, a side elevation view of a matrix subdividing around regions of scar tissue in breasts is shown in accordance with at least one embodiment. Scar tissue often forms after various surgery possibilities, including mastectomy and lumpectomy. In this embodiment, the matrix grid is subdivided in the areas of surgery. Each surgery is unique to the person, as such; this map will be generated from each scan to accurately represent the regions of the breast affected from the surgery. This subdivided grid will inform the regions of the cup that will be filled in with the multi-dimensional cellular matrix in order to best support the breasts.

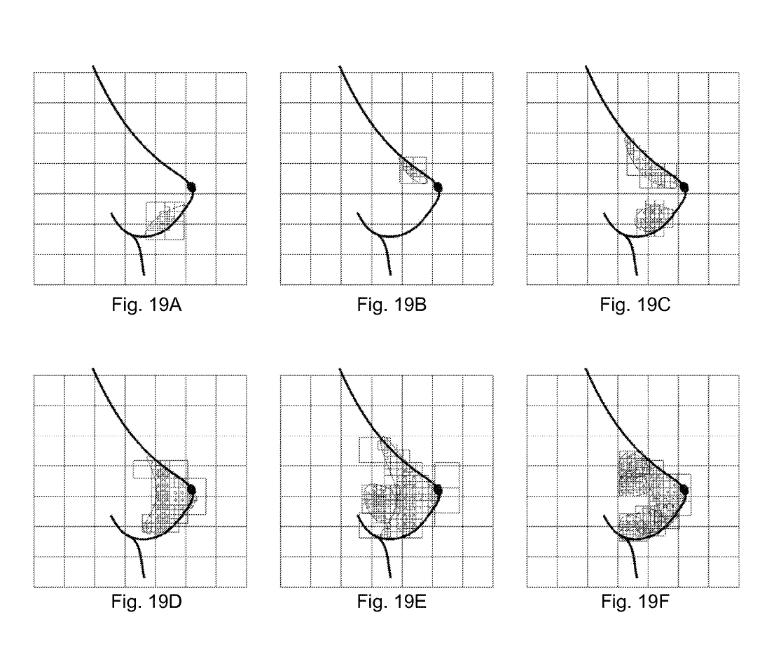

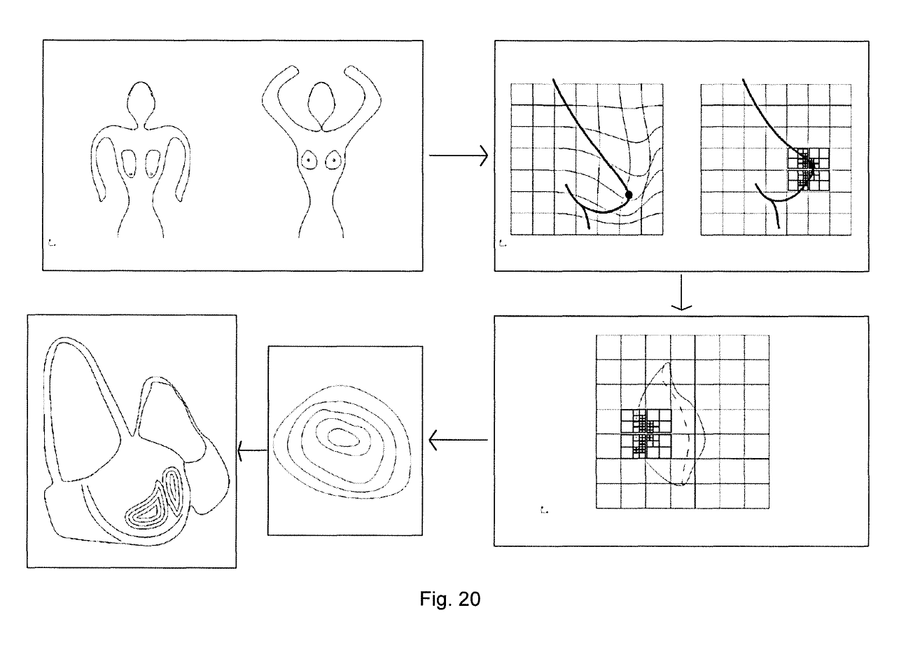

Referring to FIG. 20, some of the possible steps taken to generate the 3D custom fit brassiere are shown. In this embodiment, the steps going from 3D scan to 3D brassiere are mapped out. First, two 3D scans are taken, one with the arms down, the breasts in the relaxed position, and one with the arms up, the breasts akin to the supported position. Other scans in contemporary supported positions are also possible. The data from these scans will make up a library of breast shape translation matrices so potentially in the future, only one scan will be necessary. Next, two 3D matrices are produced, warped in the relaxed position to show the translation from relaxed position to supported position. The grid in the supported position is subdivided in the regions that underwent surgery. If the surgery happened to one breast, the grid of one breast can be compared to the grid of another. Scans may also be taken before surgery, in order to match the breast shape after surgery to before surgery. Next, the subdivided 3D matrix grid is applied to the breast cup of the breast that underwent surgery or to any abnormally shaped breast in order to locate the areas of support. Later, the multidimensional cellular matrix or 3D printed textile is thickened in the regions needing support, as per the 3D grid matrix map. This fabric can be used as either a brassiere-insert, or subsequently the multi-dimensional cellular matrix can be applied to the entire brassiere. By changing the geometry of the multi-dimensional fabric, the brassiere-straps can have more stretch than the brassiere cups. Accordingly, the entire brassiere can be fabricated in one unique, unibody, individualized brassiere apparatus.

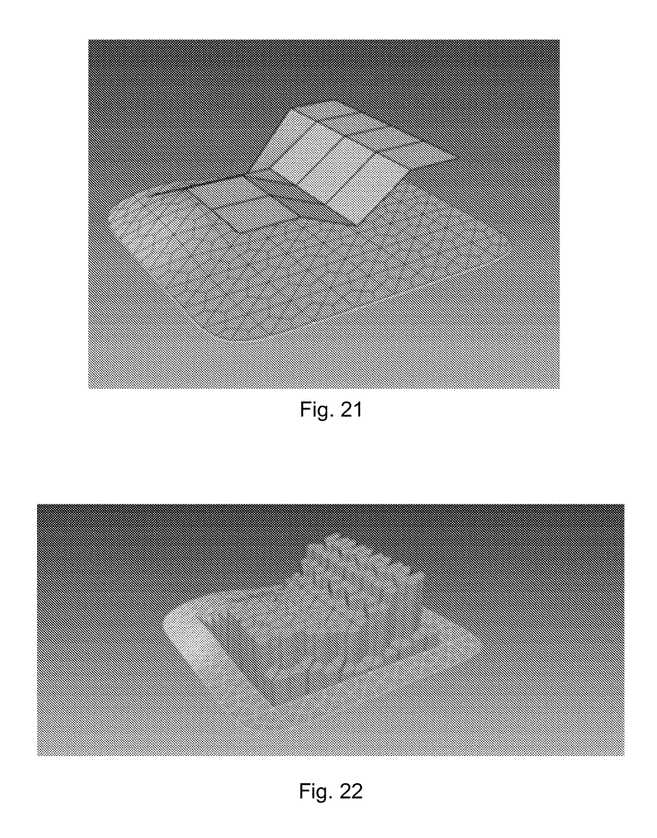

Referring to FIGS. 21 and 22, many types of computer-generated designs may be used in the central portion of the prosthesis. For example, a denser central portion, as shown in FIG. 22 may be used, or a less dense portion, as shown in FIG. 21 may be used.

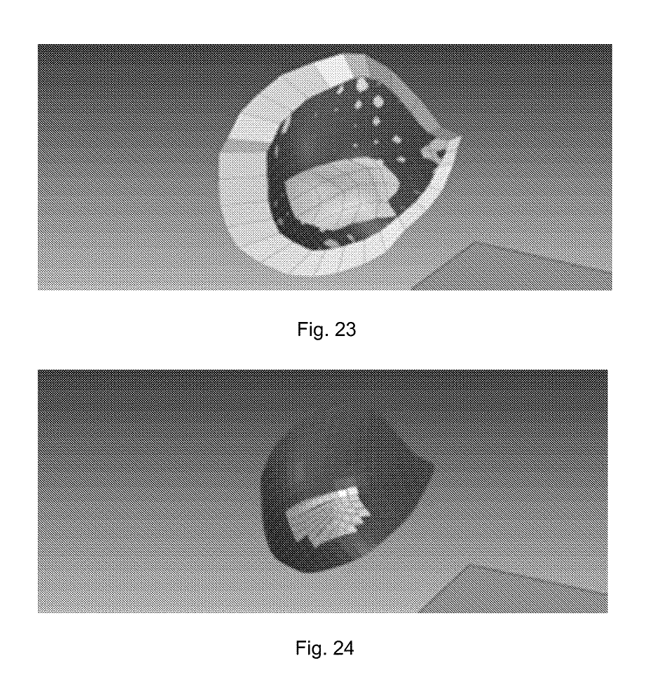

Referring to FIGS. 23 and 24, the generation step of the present disclosure may work iteratively to generate the structure for the prosthesis. FIGS. 23 and 24 illustrate one algorithm that subdivides the desired space into a mesh of a desired density and flexibility.

Referring to FIG. 25, the central structure shown between the inner wall mesh and outer wall mesh, may be formed to simulate realistic movement of human tissue. For example, in FIG. 25, it can be seen that the central portion mesh has a different structure, and density in the lower portion of the mesh as compared to the top portion. This embodiment illustrates how the mesh can be designed.

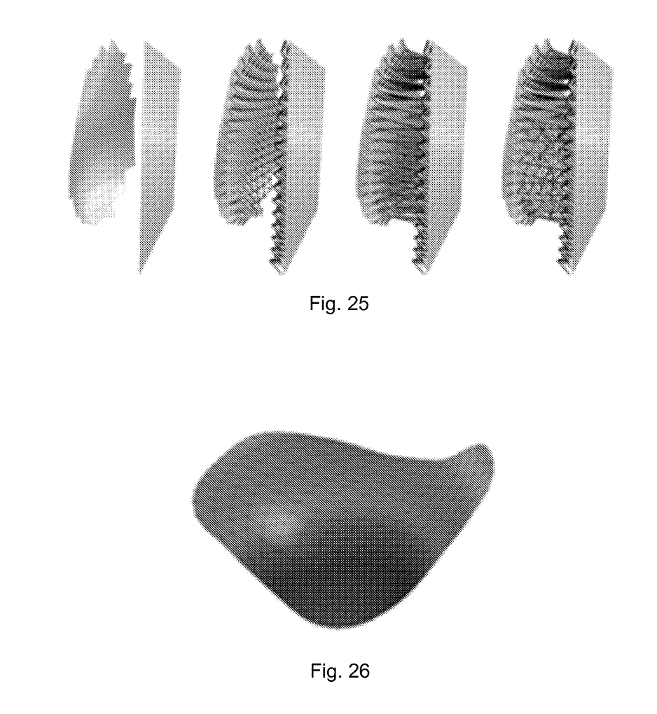

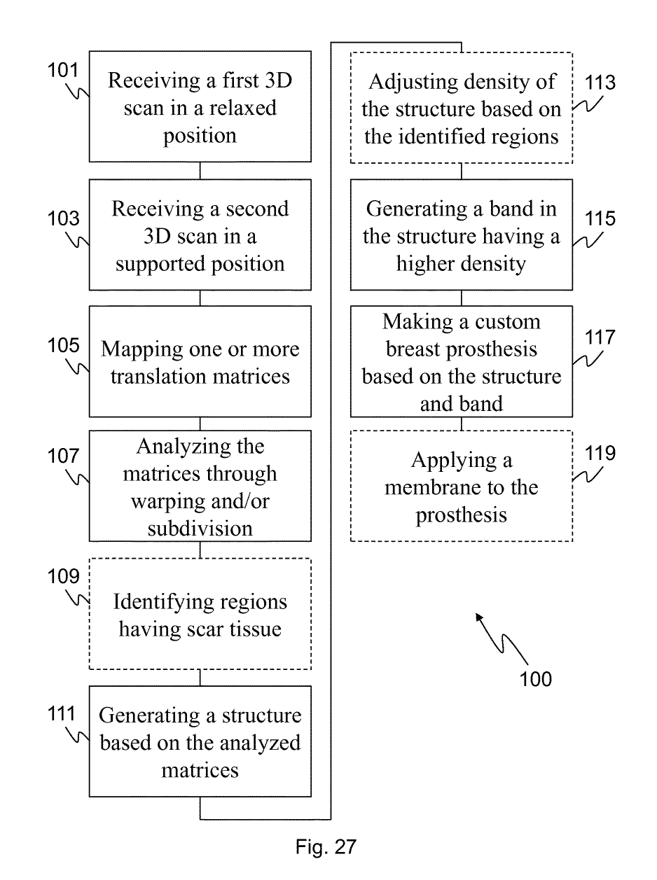

Referring to FIG. 26, this drawing illustrates, using shading, a varied density/rigidity in the generated structure. The lighter shading indicates a higher density/rigidity area and the darker shading indicates a lower density/rigidity area.

One embodiment of the present disclosure may be described as a 3D printed custom fit breast form which derives its interior and exterior envelope shape via body scan data. The interior and exterior form data may be used to define the shape to be filled. The shape may be generated as a mesh structure, for example, as described in U.S. patent application Ser. No. 14/624,578, incorporated herein by reference.

The mesh structure may be static or may have movable intersections (via interlocking or linking structures) to provide additional, life-like tissue movement. The mesh may vary in density, providing differing levels of support as needed as a traditional underwire would provide. The variation in density allows for the geometry to influence the material properties, giving at once both support and softness depending on the design of the mesh fill.

Embodiments of the present disclosure may be designed to fit line-to-line with the topography of the user's scanned body form. Embodiments may be positioned as such and worn inside a standard brassiere, however, a range of pocket-designed garments may accept the form, including, but not limited to, lingerie, swimwear, loungewear, sport or active wear.

Some embodiments of the present disclosure may be used as an implantable device, for example, to be placed subcutaneously to create a desired shape or structure of the body. In another embodiment, some embodiments of the present disclosure may be used as a scaffold for tissue regeneration. Such a scaffold may be implantable or exist ex vivo.

Some embodiments of the disclosure may include a post-processing film or other membrane addition to simulate skin-like smoothness and provide the necessary tack to hold the prosthesis in position against the body. Embodiments of the present disclosure may deliver lightweight results with superior airflow due the porous mesh nature of the structure. The prosthesis may be made from a medically approved, waterproof material.

Although the present disclosure has been described with respect to one or more particular embodiments, it will be understood that other embodiments of the present disclosure may be made without departing from the spirit and scope of the present disclosure. Hence, the present disclosure is deemed limited only by the appended claims and the reasonable interpretation thereof.

What is claimed is:

1. A breast prosthesis device for a user comprising:
an inner wall mesh having a first density, the inner wall mesh configured to align with a chest wall of the user;
an outer wall mesh having a second density, the outer wall mesh configured to have an ideal shape for the user;
a band having a density greater or equal to the first or second density; and
a central portion disposed in between the inner and outer wall meshes;
wherein the inner wall mesh, outer wall mesh, band, and central portion are electronically designed based on two or more 3D scans of the user.

2. The device of claim 1, wherein the device is formed from thermoplastic polyurethane.

3. The device of claim 1, where the central portion is filled with foam, batting, or gel.

4. The device of claim 1, wherein the inner wall mesh has fixed intersections.

5. The device of claim 1, wherein the outer wall mesh has moveable intersections.

6. The device of claim 1, further comprising a film applied to the inner wall mesh and/or outer wall mesh.

7. The device of claim 1, wherein the device is formed using a waterproof material.

8. The device of claim 1, wherein the outer wall mesh has movable linkages to provide flexibility.

9. The device of claim 1, wherein the central portion comprises several areas, each area having a density different than each other area.

10. A breast prosthesis device for a user comprising:
an inner wall mesh having a first density, the inner wall mesh having fixed intersections configured to align with a chest wall of the user and;
an outer wall mesh having a second density, the outer wall mesh having movable intersections configured to have an ideal shape for the user;
a band mesh having a density greater or equal to the first or second density; and
a central portion disposed in between the inner and outer wall meshes;
wherein the inner wall mesh is configured to align with the chest wall of the user based on two or more 3D scans of the user in one or more positions.

11. A method for making a custom breast prosthesis for a user, comprising:
receiving, from a scanner, a first 3D scan of at least a portion of the user in a relaxed position;
receiving, from the scanner, a second 3D scan of the portion of the user in a supported position;
mapping, using a processor, one or more translation matrices corresponding to the first and second 3D scans;
analyzing, using the processor, the one or more matrices through warping and/or subdivision;
generating, using the processor, a structure for the prosthesis based on the one or more analyzed matrices;
generating, using the processor, a band in the structure, the band having a higher density than the structure;
making, via additive manufacturing, a custom breast prosthesis for the user based on the generated structure and band.

12. The method of claim 11, wherein the prosthesis is made using selective laser sintering.

13. The method of claim 11, wherein analyzing the one or more matrices comprises warping the one or more matrices using a least-squares regression method.

14. The method of claim 13, further comprising analyzing the one or more matrices to identify regions of the user having scar tissue and adjusting the density of the structure based on the identified regions of the user.

15. The method of claim 11, wherein the band is generated based on the shape of the user's upper torso, back, shoulder, or ribcage.

16. The method of claim 11, wherein the supported position comprises the user's arms held straight up.

17. The method of claim 11, further comprising applying a membrane to at least a portion of the prosthesis.

18. The method of claim 11, wherein the generated structure is flexible in three dimensions.

19. The method of claim 11, wherein the generated structure is porous.

20. The method of claim 11, further comprising:
receiving, from the scanner, a third 3D scan of an ideal portion of the user in a relaxed position;
receiving, from the scanner, a fourth 3D scan of the ideal portion of the user in a supported position;
mapping, using the processor, one or more translation matrices corresponding to the third and fourth 3D scans; and
comparing, using the processor, the one or more translation matrices corresponding to the third and fourth 3D scans with the one or more translation matrices corresponding to the first and second 3D scans;
wherein, the generated structure and generated band are based on the compared translation matrices.

21. The method of claim 11, wherein the generated structure has moveable linkages to provide flexibility.

22. The method claim 11, wherein the structure comprises several areas, each area having a density different than each other area.

23. A breast prosthesis device for a user comprising:
an inner mesh portion having a first density, the inner mesh portion configured to align with a chest wall of the user;
an outer mesh portion having a second density, the outer mesh portion configured to have an ideal shape for the user; and
a band having a density greater or equal to the first or second density;
wherein the inner mesh portion, outer mesh portion, and band are electronically designed based on two or more 3D scans of the user.
